SEQUENCE LISTING (3 sequences)

SEQ ID NO: 1 - protein, 597 aa, artificial
  SHP2  AA sequence Isoform 1
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLADQTSGDQSPLPPCTPTPPCAEMREDSARVYENVGLMQQQKSFR

SEQ ID NO: 2 - protein, 593 aa, artificial [sequence omitted]

SEQ ID NO: 3 - protein, 460 aa, artificial
  SHP2 AA sequence Isoform 3
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQGNTERTVWQYHFRTWPDHGVPSDPGGVLDFLEEVHHKQESIMDAGPVVVHCR

(19) United States
(12) Patent Application Publication
(10) Pub. No.: US 2021/0230300 A1
(43) Pub. Date: Jul. 29, 2021

(54) INHIBITORS OF SHP2

(71) Applicant: Bayer Aktiengesellschaft, Leverkusen (DE)

(72) Inventors: Ulf BRÜGGEMEIER, Leichlingen (DE); Tibor SCHOMBER, Iserlohn (DE); Karoline DRÖBNER, Velbert (DE); David ENGEL, Wuppertal (DE); Michael BECKER, Solingen (DE)

(73) Assignee: Bayer Aktiengesellschaft, Leverkusen (DE)

(21) Appl. No.: 16/972,404

(22) PCT Filed: May 28, 2019
(86) PCT No.: PCT/EP2019/063736

§ 371 Date: Dec. 4, 2020

(30) Foreign Application Priority Data
Jun. 4, 2018 (EP) 18175742.8

(51) Int. Cl. C07K 16/40; A61P 13/12; C12Q 1/6876; C12Q 1/6813

(52) U.S. Cl. CPC C07K 16/40; A61P 13/12; C07K 2317/76; C12Q 1/6813; C12Q 1/6876

(57) ABSTRACT

The present invention is related to an inhibitor or antagonist of SHP2 for the treatment and/or prevention of a neoplastic disease.

The present invention provides allosteric inhibitors and antagonists of SHP2, for the treatment and/or prevention of a kidney disease. The invention provides inhibitors or antagonists in the form of antibodies, fragments and derivatives thereof, antibody mimetics, nucleic acids, aptamers, or small molecules. The invention also provides assays and screening technologies to find such antagonists or inhibitors.

### BACKGROUND OF THE INVENTION

Chronic Kidney Disease (CKD) is a type of kidney disease in which there is gradual loss of kidney function over a period of months or years. Early on there are typically no symptoms. Later, leg swelling, feeling tired, vomiting, loss of appetite, or confusion may develop. Complications may include heart disease, high blood pressure, bone disease, or anemia.

Causes of kidney disease include diabetes, high blood pressure, glomerulonephritis, and polycystic kidney disease, as well as exposure to X ray contrast media and cytotoxic agents, like cisplatin. Risk factors include a family history of the condition. Diagnosis is generally by blood tests to measure the glomerular filtration rate and urine tests to measure albumin. Further tests such as an ultrasound or kidney biopsy may be done to determine the underlying cause. A number of different classification systems exist.

Apart from controlling other risk factors, the goal of therapy is to slow down or halt the progression of CKD. Control of blood pressure and treatment of the original disease are the broad principles of management.

Generally, angiotensin converting enzyme inhibitors (ACEIs) or angiotensin II receptor antagonists (ARBs) are used, as they have been found to slow the progression. They have also been found to reduce the risk of major cardiovascular events such as myocardial infarction, stroke, heart failure, and death from cardiovascular disease when compared to placebo in individuals with CKD. Furthermore, ACEIs may be superior to ARBs for protection against progression to kidney failure and death from any cause in those with CKD. Aggressive blood pressure lowering decreases peoples' risk of death. Although the use of ACE inhibitors and ARBs represents the current standard of care for people with CKD, people progressively lose kidney function while on these medications, which reported a decrease over time in estimated GFR (an accurate measure of CKD progression, as detailed in the K/DOQI guidelines) in people treated by these conventional methods.

Aggressive treatment of high blood lipids has also been recommended. Low-protein, low-salt diet may result in slower progression of CKD and reduction in proteinuria as well as controlling symptoms of advanced CKD to delay dialysis start. Replacement of erythropoietin and calcitriol, two hormones processed by the kidney, is often necessary in people with advanced disease. Guidelines recommend treatment with parenteral iron prior to treatment with erythropoietin. A target hemoglobin level of 9-12 g/dL is recommended. The normalization of hemoglobin has not been found to be of benefit. It is unclear if androgens help with anemia. Phosphate binders are also used to control the serum phosphate levels, which are usually elevated in advanced Chronic Kidney Disease. Although the evidence for them is limited, phosphodiesterase-5 inhibitors and zinc show potential for helping men with sexual dysfunction.

At stage 5 CKD, renal replacement therapy is usually required, in the form of either dialysis or a transplant.

CKD increases the risk of cardiovascular disease, and people with CKD often have other risk factors for heart disease, such as high blood lipids. The most common cause of death in people with CKD is cardiovascular disease rather than kidney failure.

Chronic Kidney Disease results in worse all-cause mortality (the overall death rate) which increases as kidney function decreases. The leading cause of death in Chronic Kidney Disease is cardiovascular disease, regardless of whether there is progression to stage 5.

While renal replacement therapies can maintain people indefinitely and prolong life, the quality of life is negatively affected. Kidney transplantation increases the survival of people with stage 5 CKD when compared to other options; however, it is associated with an increased short-term mortality due to complications of the surgery. Transplantation aside, high-intensity home hemodialysis appears to be associated with improved survival and a greater quality of life, when compared to the conventional three-times-a-week hemodialysis and peritoneal dialysis.

Patients with end-stage kidney disease (ESKD) are at increased overall risk for cancer. This risk is particularly high in younger patients and gradually diminishes with age.

Therefore, it is one object of the present invention to improve treatment options for kidney disease. It is another object of the present invention to provide alternative treatment options for kidney disease.

### SUMMARY OF THE INVENTION

These and further objects are met with methods and means according to the independent claims of the present invention. The dependent claims are related to specific embodiments.

### EMBODIMENTS OF THE INVENTION

Before the invention is described in detail, it is to be understood that this invention is not limited to the particular component parts or structural features of the devices or compositions described or process steps of the methods described as such devices and methods may vary. It is also to be understood that the terminology used herein is for purposes of describing particular embodiments only, and is not intended to be limiting. The mere fact that certain measures are recited in mutually different dependent claims does not indicate that a combination of these measures cannot be used to advantage. Any reference signs in the claims should not be construed as limiting the scope. It must be noted that, as used in the specification and the appended claims, the singular forms “a,” “an” and “the” include singular and/or plural referents unless the context clearly dictates otherwise. Further, in the claims, the word “comprising” does not exclude other elements or steps. The mere fact that certain measures are recited in mutually different dependent claims does not indicate that a combination of these measures cannot be used to advantage.

It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the length of this specification manageable. It is further to be understood that the content of the prior art documents referred to herein is incorporated by reference, e.g., for enablement purposes, namely when e.g. a method is discussed details of which are described in said prior art document. This approach serves to keep the length of this specification manageable.

According to one aspect of the invention, an inhibitor or antagonist of SHP2 for the treatment and/or prevention of a kidney disease is provided.

The term “kidney disease”, as used herein, relates to diseases and/or conditions associated with chronic kidney disease (CKD), Diabetic Kidney Disease (DKD) (Lyo et al., 2012) and renal disorders, in particular acute and chronic renal insufficiency. For the purpose of the present invention the term “renal insufficiency” comprises both acute and chronic manifestations of renal insufficiency, and also underlying or related renal disorders such as diabetic (Liu, 2006) and non-diabetic nephropathies, hypertensive nephropathies, ischaemic renal disorders (Chihanga, 2018), renal hypoperfusion, intradialytic hypotension, obstructive uropathy, renal stenoses, glomerulopathies (Zhu, 2013), glomerulonephritis (such as, for example, primary glomerulonephritides; minimal change glomerulonephritis (lipoidnephrosis); membranous glomerulonephritis; focal segmental glomerulosclerosis (FSGS); membrane-proliferative glomerulonephritis; crescentic glomerulonephritis; mesangioproliferative glomerulonephritis (IgA nephritis (Zhu, 2013), Berger's disease); post-infectious glomerulonephritis; secondary glomerulonephritides: diabetes mellitus, lupus erythematosus, amyloidosis, Goodpasture syndrome, Wegener granulomatosis, Henoch-Schönlein purpura, microscopic polyangiitis, acute glomerulonephritis, pyelonephritis (for example as a result of: urolithiasis, benign prostate hyperplasia, diabetes, malformations, abuse of analgesics, Crohn's disease), glomerulosclerosis, arteriolonecrose of the kidney, tubulointerstitial diseases, nephropathic disorders such as primary and congenital or acquired renal disorder, Alport syndrome, nephritis, immunological kidney disorders such as kidney transplant rejection and immunocomplex-induced renal disorders, nephropathy induced by toxic substances, nephropathy induced by contrast agents, diabetic and non-diabetic nephropathy, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerular and arterioles, tubular dilatation, hyperphosphataemia and/or the need for dialysis. The present invention also comprises the use of the compounds according to the invention for the treatment and/or prophylaxis of sequelae of renal insufficiency, for example pulmonary edema, heart failure, uremia, anemia, as well as for chronic allograft nephropathy and polycystic kidney disease.

SHP2 (Tyrosine-protein phosphatase non-receptor type 11, UniProtKB-Q06124), also known as PTPN11, protein-tyrosine phosphatase 1D (PTP-1D), SHP-2, or protein-tyrosine phosphatase 2C (PTP-2C) is an enzyme that in humans is encoded by the PTPN11 gene. PTPN11 is a protein tyrosine phosphatase (PTP) Shp2.

PTPN11 is a member of the protein tyrosine phosphatase (PTP) family. PTPs are known to be signaling molecules that regulate a variety of cellular processes including cell growth, differentiation, mitotic cycle, and oncogenic transformation. This PTP contains two tandem Src homology-2 domains, which function as phospho-tyrosine binding domains and mediate the interaction of this PTP with its substrates. This PTP is widely expressed in most tissues and plays a regulatory role in various cell signaling events that are important for a diversity of cell functions, such as mitogenic activation, metabolic control, transcription regulation, and cell migration. Mutations in this gene are a cause of Noonan syndrome as well as acute myeloid leukemia.

This phosphatase, along with its paralogue, SHP1, possesses a domain structure that consists of two tandem SH2 domains in its N-terminus followed by a protein tyrosine phosphatase (PTP) domain. In the inactive state, the N-terminal SH2 domain binds the PTP domain and blocks access of potential substrates to the active site. Thus, Shp2 is auto-inhibited.

Upon binding to target phospho-tyrosyl residues, the N-terminal SH2 domain is released from the PTP domain, catalytically activating the enzyme by relieving this auto-inhibition.

SHP2 is expressed in 3 isoforms as shown in the following table:

| | UniProt | | SEQ ID | |
|---|---|---|---|---|
| Isoform | Identifier | Alias | NO | Remarks |
| 1 | Q06124-1 | PTP2Ci | 1 | This isoform is often referred |
| | | | | to as the ‘canonical’ sequence. |
| 2 | Q06124-2 | PTP2C | 2 | The sequence of this isoform |
| | | | | differs from the canonical |
| | | | | sequence as follows: |
| | | | | 408-411 Missing. |
| 2 | Q06124-3 | | 3 | The sequence of this isoform |
| | | | | differs from the canonical |
| | | | | sequence as follows: |
| | | | | 408-411: Missing |
| | | | | 464-464: → R |
| | | | | 465-597: Missing. |

There is prior art which suggests a functional relationship between SHP2 and kidney conditions. Saxton et al. (1997) have shown that a functional knockout of SHP2, by deletion of 65 AA in the N-terminus of SHP2, is lethal, hence, putting a systemic administration of an SHP2 inhibitor into question.

David et al. (2010) have disclosed results in which a heterozygous knockout mouse that demonstrates a 50% reduction of SHP2 expression in all tissues (heterozygous null mutant SHP2^{+/-}). Comparison with wildtype mice shown that loss of 50% gene/protein dosage of PTPN11/SHP2 was insufficient to affect glomerular (and thereby nephron) number in mouse kidneys in vivo. These results, again, put the potential efficacy of a systemic administration of an SHP2 inhibitor into question.

Wang et al. (2016) have shown that an unspecific, non-allosteric SHP2 Inhibitor exhibits activity in a murine SLE (systematic Lupus erythematosus)-mediated autoimmune disease model. Treatment of MRL/lpr mice with the SHP2 inhibitor prevented the progression of kidney disease in SLE-prone mice.

The hydroxyindole carboxylic acid-based SHP2 inhibitor (called 11a-1) anchors to the SHP2 active site, with strong potency (IC50 200 nM) and selectivity (>5-fold against any of 20 other PTPs). The inhibitor is disclosed in WO2015003094 and has the formula

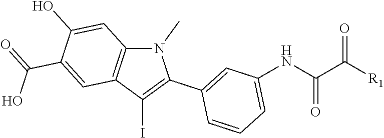

wherein Ri=NRaRb, wherein Ra or Rb can each independently be selected from the group consisting of hydrogen, unsubstituted or substituted alkyl, unsubstituted or substituted cycloalkyl, unsubstituted or substituted heterocyclyl, unsubstituted or substituted aryl, unsubstituted or substituted heteroaryl, and unsubstituted or substituted fused 5-12 member aromatic or aliphatic ring system, wherein the substitution on the fused 5-12 member aromatic or aliphatic ring system is selected from the group consisting of nitrogen, oxygen and sulfur.

Attenuation of renal fibrosis in conditional tissue specific SHP2 knockout mice was published by Teng et al (2015). A tissue specific knockout animal does yet not reflect the activity of a systemically administered compound inhibiting SHP2 in all tissues.

According to one embodiment of the present invention, the inhibitor or antagonist is an allosteric inhibitor or antagonist. As used herein, the term “allosteric inhibitor” or “allosteric antagonist” relates to an agent that, by binding to an allosteric site of a target protein, alters the protein conformation in the active site of the target, and, consequently changes the shape of active site. Thus, the target, e.g., an enzyme, no longer remains able to bind to its specific substrate, or experiences a reduced ability to bind its substrate.

Currently, three allosteric binding sites in SHP are known, as shown in the following table:

| No | Nickname | Defined by | Reference |
|---|---|---|---|
| 1 | “tunnel” | Q495, L254 P491, | Fodor et al (2018) |
| | | E250, F113, | |
| | | R111, Q257, | |
| 2 | “latch” | N92 - V95, H85- | Fodor et al (2018) |
| | | E90, E83, R265 | |
| 3 | N-SH2/PTP | C333-C367 | Chio et al (2015) |
| | domain | | |
| | interface. | | |

However, the use of an allosteric inhibitor or allosteric antagonist of SHP2 in the treatment of Chronic Kidney Disease has so far not been disclosed.

According to one embodiment of the present invention, the inhibitor or antagonist is a monoclonal antibody, or a target-binding fragment or derivative thereof retaining target binding capacities, or an antibody mimetic, which specifically binds to the SHP2 protein.

As used herein, the term “monoclonal antibody (mAb)”, shall refer to an antibody composition having a homogenous antibody population, i.e., a homogeneous population consisting of a whole immunoglobulin, or a fragment or derivative thereof retaining target binding capacities. Particularly preferred, such antibody is selected from the group consisting of IgG, IgD, IgE, IgA and/or IgM, or a fragment or derivative thereof retaining target binding capacities.

As used herein, the term “fragment” shall refer to fragments of such antibody retaining target binding capacities, e.g.
a CDR (complementarity determining region)
a hypervariable region,
a variable domain (Fv)
an IgG heavy chain (consisting of VH, CH1, hinge, CH2 and CH3 regions)
an IgG light chain (consisting of VL and CL regions), and/or
a Fab and/or F(ab)₂.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs, and further retaining target binding capacities. All these items are explained below.

Other antibody derivatives known to the skilled person are Diabodies, Camelid Antibodies, Nanobodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerised constructs comprising CH3+VL+VH, and antibody conjugates (e.g. antibody or fragments or derivatives linked to a toxin, a cytokine, a radioisotope or a label). These types are well described in literature and can be used by the skilled person on the basis of the present disclosure, with adding further inventive activity.

As discussed above, SHP2 is sufficiently specified to enable a skilled person to make a monoclonal antibody thereagainst. Routine methods encompass hybridoma, chimerization/humanization, phage display/transgenic mammals, and other antibody engineering technologies.

Methods for the production of a hybridoma cell are disclosed in Köhler & Milstein (1975). Essentially, e.g., a mouse is immunized with a human SHP2 protein, following B-cell isolation and fusion with a myeloma cell.

Methods for the production and/or selection of chimeric or humanised mAbs are known in the art. Essentially, e.g., the protein sequences from a murine anti SHP2 antibody which are not involved in target binding are replaced by corresponding human sequences. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies.

Methods for the production and/or selection of fully human mAbs are known in the art. These can involve the use of a transgenic animal which is immunized with human SHP2, or the use of a suitable display technique, like yeast display, phage display, B-cell display or ribosome display, where antibodies from a library are screened against human SHP2 in a stationary phase.

In vitro antibody libraries are, among others, disclosed in U.S. Pat. No. 6,300,064 by Morph® Sys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in EP1480515A2 by TaconicArtemis. IgG, scFv, Fab and/or F(ab)₂ are antibody formats well known to the skilled person. Related enabling techniques are available from the respective textbooks.

As used herein, the term “Fab” relates to an IgG fragment comprising the antigen binding region, said fragment being composed of one constant and one variable domain from each heavy and light chain of the antibody

As used herein, the term “F(ab)₂” relates to an IgG fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually serine (S) or glycine (G). This chimeric molecule retains the specificity of the original immunoglobulin, despite removal of the constant regions and the introduction of a linker peptide. Modified antibody formats are for example bi- or trispecific antibody constructs, antibody-based fusion proteins, immunoconjugates and the like. These types are well described in literature and can be used by the skilled person on the basis of the present disclosure, with adding further inventive activity. Finding a suitable antibody, or fragment or derivative, that is capable of acting as an inhibitor or antagonist of SHP2, e.g., by binding to its active center, is hence a matter of routine for the skilled person, based on the public availability of the amino acid sequences of the different SHP2 isoforms. Polyclonal antibodies against SHP2 for scientific research are commercially available, e.g., from Abcam (ab32083, ab131541, ab10555), Rockland Immunochemicals (600-401-EJ6) or EMD Millipore (06-118), emphasizing that the skilled person is capable of also making therapeutic antibodies against said target.

As used herein, the term “antibody mimetic” relates to an organic molecule, most often a protein that specifically binds to a target protein, similar to an antibody, but is not structurally related to antibodies. Antibody mimetics are usually artificial peptides or proteins with a molar mass of about 3 to 20 kDa. The definition encompasses, inter alia, Affibody molecules, Affilins, Affimers, Affitins, Alphabodies, Anticalins, Avimers, DARPins, Fynomers, Kunitz domain peptides, Monobodies, and nanoCLAMPs.

Because SHP2 is an intracellular target, the antibody or its fragment or derivative, or the antibody mimetic, needs to be funneled or trafficked into the intracellular space. Routine technologies are available for this purposes, which are disclosed, inter alia, in Chen & Erlanger (2002), Berguig et al (2015).

According to one embodiment of the present invention, the inhibitor or antagonist comprises a first nucleic acid molecule that specifically binds to a second nucleic acid molecule, which second nucleic acid molecule encodes for the SHP2 protein.

Said second nucleic acid molecule can be an mRNA transcribed from the gene encoding for the SHP2 protein. Said second nucleic is devoid of introns, but due to alternative splicing different mRNAs transcribed from the gene encoding for the SHP2 protein can differ from one another. In such case, the first nucleic acid molecule can be a siRNA (small interfering RNA) or a shRNA (short hairpin RNA). siRNAs are short artificial RNA molecules which can be chemically modified to enhance stability. Because siRNAs are double-stranded, the principle of the ‘sense’ and the ‘antisense’ strand also applies. The sense strands have a base sequence identical to that of the transcribed mRNA and the antisense strand has the complementary sequence. Technically, a siRNA molecule administered to a patient is bound by an intracellular enzyme called Argonaut to form a so-called RNA-induced silencing complex (RISC). The antisense strand of the siRNA guides RISC to the target mRNA, where the antisense strand hybridizes with the target mRNA, which is then cleaved by RISC. In such way, translation of the respective mRNA is interrupted. The RISC can then cleave further mRNAs. Delivery technologies are e.g. disclosed in Xu and Wang (2015). Finding a suitable sequence for the siRNA is a matter of routine for the skilled person, based on the public availability of the different mRNA isoforms of SHP2. shRNA is an artificial RNA molecule with a tight hairpin turn that can be used to silence target gene expression via RNA interference (RNAi). shRNA can be delivered to cells, e.g., by means of a plasmid or through viral or bacterial vectors. shRNA is an advantageous mediator of RNAi in that it has a relatively low rate of degradation and turnover. The respective plasmids comprise a suitable promoter to express the shRNA, like a polymerase III promoter such as U6 and H1 or a polymerase II promoter. Once the plasmid or vector has integrated into the host genome, the shRNA is transcribed in the nucleus. The product mimics pri-microRNA (pri-miRNA) and is processed by Drosha. The resulting pre-shRNA is exported from the nucleus by Exportin 5. This product is then processed by Dicer and loaded into the RNA-induced silencing complex (RISC), after which the same silencing follows as in siRNA. Finding a suitable sequence for the shRNA is a matter of routine for the skilled person, based on the public availability of the different mRNA isoforms of SHP2.

Said second nucleic acid molecule can also be a genomic DNA comprised in the gene encoding for the SHP2 protein. Said gene comprises several non-coding introns, hence its sequence differs from the sequence of the mRNA or the cDNA disclosed herein.

In such case, the first nucleic acid molecule can be the guide RNA of a CRISPR Cas system (see, e.g., Jinek et al (2012)), which guide RNA comprises a target-specific crRNA (“small interfering CRISPR RNA”) capable of hybridizing with a genomic strand of the SHP2 gene (or, the first nucleic acid molecule can be the crRNA alone). The guide RNA/crRNA is capable of directing the Cas enzyme, which is an endonuclease, to the SHP2 gene, where the Cas enzyme carries out sequence specific strand breaks. By creating one or more double strand breaks, the SHP2 gene hence can be silenced. To use said system for in vivo gene silencing of SHP2, e.g., in different cells of a tumor, a dedicated delivery technology is required, which comprise a delivery vehicle such as lipid nanoparticles, as for example discussed in Yin et al (2016). Finding a suitable sequence for the crRNA comprised in the guide RNA is a matter of routine for the skilled person, based on the public availability of the genomic sequence of the SHP2 gene.

In another embodiment, said first nucleic acid molecule can also the guide RNA of a CRISPR Cpf system (Zetsche et al (2015)), which guide RNA comprises a target-specific crRNA (“small interfering CRISPR RNA”). Similar to CRISPR Cas, the guide RNA is capable of directing the Cpf enzyme, which is an endonuclease, to the SHP2 gene. As regards technical considerations, e.g., delivery for in vivo applications and finding of the suitable sequence for the first nucleic acid molecule, similar aspects as with CRISPR Cas apply.

Further embodiments of the CRISPR technology are currently under development, with different endonucleases. However, all these approaches use a target-specific RNA (the guide RNA or crRNA as in CRISPR Cas) that hybridizes with a target sequence. In all these cases, the target-specific RNA qualifies as the first nucleic acid molecule in the meaning of the preferred embodiment discussed herein. As regards technical considerations, e.g., delivery for in vivo applications and finding of the suitable sequence for the first nucleic acid molecule, similar aspects as with CRISPR Cas apply.

According to one embodiment of the present invention, the antagonist or inhibitor is an aptamer that specifically binds to the SHP2 protein.

Aptamers are oligonucleotides that have specific binding properties for a pre-determined target. They are obtained from a randomly synthesized library containing up to 10¹⁵ different sequences through a combinatorial process named SELEX (“Systematic Evolution of Ligands by EXponential enrichment”). Aptamer properties are dictated by their 3D shape, resulting from intramolecular folding, driven by their primary sequence. An aptamer3D structure is exquisitely adapted to the recognition of its cognate target through hydrogen bonding, electrostatic and stacking interactions. Aptamers generally display high affinity (K_{d} about micromolar for small molecules and picomolar for proteins).

An overview on the technical repertoire to generate target specific aptamers is given, e.g., in Blind and Blank (2015). Aptamers can also be delivered into the intracellular space, as disclosed in Thiel & Giangrande (2010).

Finding a suitable aptamer that is capable of acting as an inhibitor or modulator of SHP2, e.g., by binding to its active centre or an allosteric site, is hence a matter of routine for the skilled person, based on the public availability of the amino acid sequences of the different SHP2 isoforms.

According to one embodiment of the present invention, the antagonist or inhibitor is a small molecule that specifically binds to one or more isoforms of the SHP2 protein.

Small molecular allosteric inhibitors of SHP2 have already been described in the scientific literature already, yet not in the treatment of Chronic Kidney Disease.

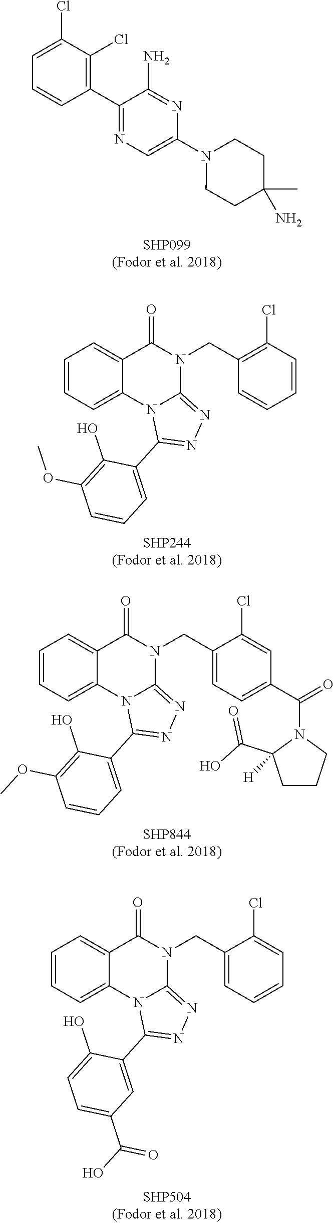

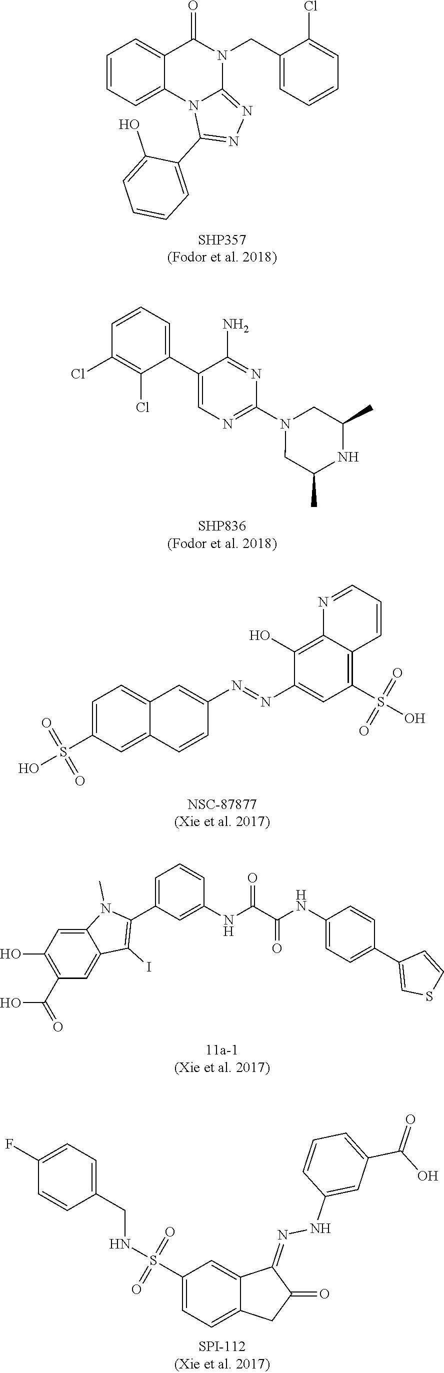 [SMILES: O=c1c2ccccc2n2c(-c3ccccc3O)nnc2n1Cc1ccccc1Cl ; C[C@@H]1CN(c2ncc(-c3cccc(Cl)c3Cl)c(N)n2)C[C@H](C)N1 ; O=S(=O)(O)c1ccc2cc(/N=N/c3cc(S(=O)(=O)O)c4cccnc4c3O)ccc2c1 ; Cn1c(-c2cccc(NC(=O)C(=O)Nc3ccc(-c4ccsc4)cc3)c2)c(I)c2cc(C(=O)O)c(O)cc21 ; O=C1Cc2ccc(S(=O)(=O)NCc3ccc(F)cc3)cc2/C1=N/Nc1cccc(C(=O)O)c1]

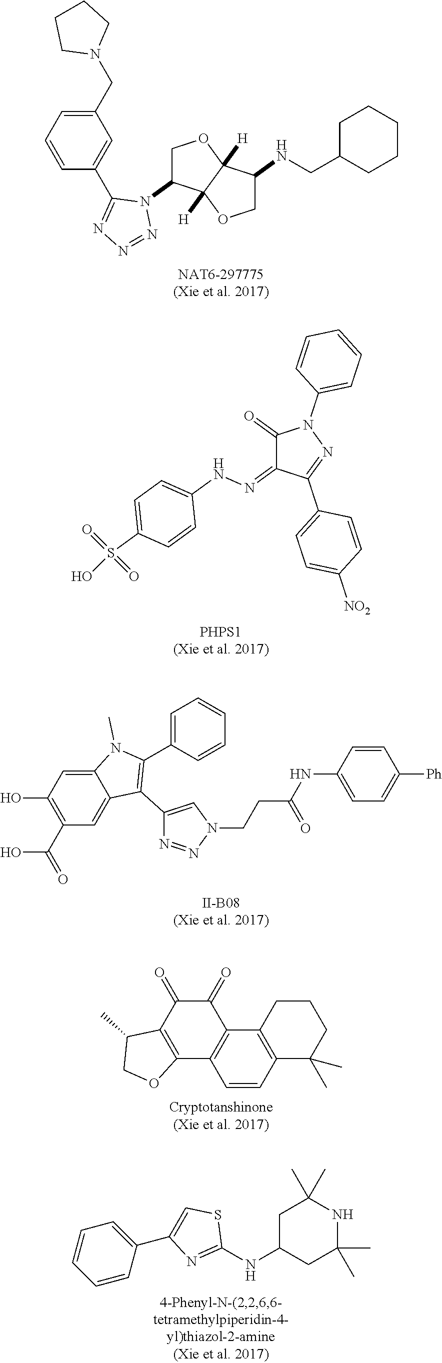

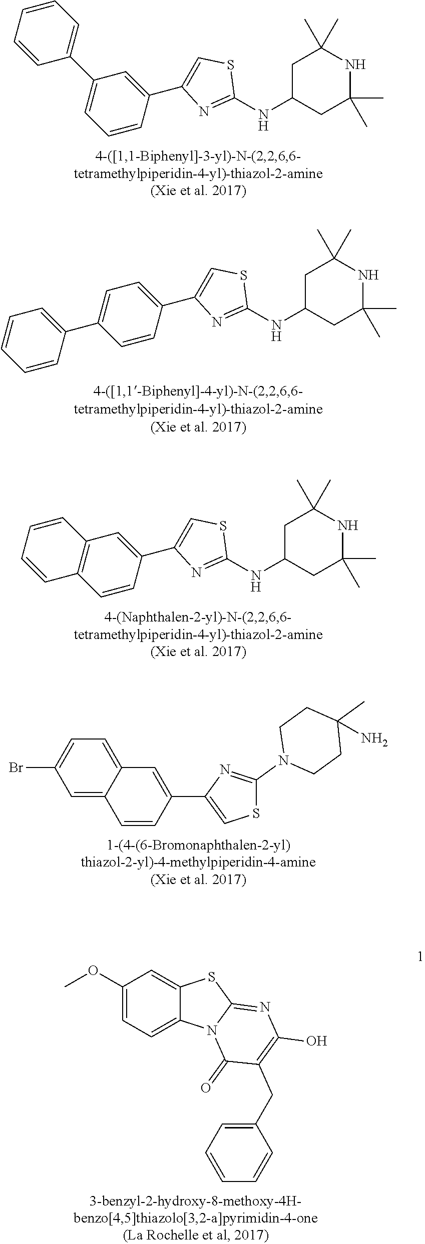

All of these molecules have the potential to act as inhibitors or antagonists of SHP2 for the treatment and/or prevention of kidney diseases.

According to one embodiment of the present invention, the antagonist or inhibitor can be found by means of a SHP2 inhibition assay.

According to one embodiment of the present invention, the SHP2 protein to which the antibody, fragment or derivative, antibody mimetic, aptamer or small molecule binds comprises a sequence comprised in any of SEQ IDs No 1-3.

According to one embodiment of the present invention, the second nucleic acid molecule encoding the SHP2 protein comprises a sequence comprised in SEQ ID No 2.

According to another aspect of the invention, the use of an inhibitor or antagonist according to the above description (for the manufacture of a medicament) in the treatment of a human or animal subject
being diagnosed for,
suffering from or
being at risk of

developing a kidney diseases is provided.

According to another aspect of the invention, a pharmaceutical composition comprising an inhibitor or antagonist according to the above description is provided.

According to another aspect of the invention, a combination of a pharmaceutical composition according to the above description and one or more therapeutically active compounds is provided.

According to another aspect of the invention, a method for treating or preventing a kidney disease is provided, comprising administering to a subject in need thereof an effective amount of the inhibitor or antagonist, the pharmaceutical composition according or the combination according to the above description.

In one embodiment, the kidney disease is characterized by overactivity or overexpression of SHP2. The term “overactivity”, as used herein, means a change in the level of SHP2 protein activity, compared to a healthy, non pathologic tissue of the same type of tissue, under analogous conditions. Preferably, said change is at least 20% above the level in a healthy, non pathologic tissue of the same type of tissue, more preferably at least 30, 40, 50, 60, 70, 80, 90, 100, 150, 200, 300, 400, 500, 600, 700, 800, 900, 100 or even 2000% above that level.

The term “overexpression”, as used herein, means a change in the level of SHP2 protein or SHP2 mRNA, compared to a healthy, non pathologic tissue of the same type of tissue, under analogous conditions. Preferably, said change is at least 20% above the level in a healthy, non pathologic tissue of the same type of tissue, more preferably at least 30, 40, 50, 60, 70, 80, 90, 100, 150, 200, 300, 400, 500, 600, 700, 800, 900, 100 or even 2000% above that level.

According to another aspect of the invention, a method for identifying a compound for use in the treatment and/or prevention of a patient suffering from, at risk of developing, and/or being diagnosed for a kidney disease, which method comprises the screening of one or more test compounds in a SHP2 inhibition assay.

According to one embodiment, such method further comprises a prior step of creation and/or provision of a library of test compounds.

According to another aspect of the invention, a method for determining whether a human or animal subject is suitable of being treated with an antagonist or inhibitor, a composition or a combination according to the above description, said method comprising
providing a tissue or liquid sample from said subject, and
   determining whether or not said sample is characterized by expression or overexpression of SHP2.

Said sample is preferably a blood or urine sample.

According to one embodiment, the expression of SHP2 is determined
on an mRNA level (e.g., RT-PCR, in situ PCR and/or Fluorescence in situ hybridization (FISH)
on a protein level (e.g., with Immunohistochemistry, Immunoblot, ELISA, and the like), and/or
on a genomic level (e.g., by sequencing of blood cells).

According to one embodiment, the SHP2 protein phosphatase activity is determined as
phosphatase activity from protein lysates, directly or after precipitation of SHP2 protein by immune precipitation
analysis of downstream effectors. The downstream effectors could be any protein or RNA modulated by SHP2 activity. Examples for known protein downstream effectors of SHP2 are the phosphorylation and dephosphorylation of ERK and other phosphorylated proteins.

According to another aspect of the invention, a companion diagnostic for use in a method according to the above description is provided, which companion diagnostic comprises at least one agent is selected from the group consisting of a nucleic acid probe or primer capable of hybridizing to a nucleic acid (DNA or RNA) that encodes an SHP2 protein
an antibody that is capable of binding to a SHP2 protein, and/or
an aptamer that is capable of binding to a SHP2 protein

SHP2 Inhibition Assay

SHP2 (R&D Systems) has been activated through a bisphorphorylated peptide. The activation of the enzyme was inhibited by test compounds. The catalytic activity of SHP2 was monitored using the fluorescence substrate DiFMUP. The reactions were performed at room temperature in a 1536-well white polystyrene plate. In a volume of 4 μl 50 mM HEPES, pH 7.2, 50 mM NaCl, 1 mM EDTA, 0.05% Tween, 5 mM DTT, 10 μM DIFMUP, 0.2 nM of SHP2 was co-incubated with of 1 μM of bisphosphorylated IRS1 peptide (sequence: H2N-LN(pY)IDLDLV(dPEG8)LST(pY)ASINFQK-amide, JPT Peptide Technology) and inhibitory compounds. The fluorescence signal was monitored using a microplate reader (BMG Pherastar) using excitation and emission wavelengths of 340 nm and 450 nm, respectively. The inhibitor dose-response curves were analysed using GraphPad Software.

### EXPERIMENTS AND FIGURES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Any reference signs should not be construed as limiting the scope. All amino acid sequences disclosed herein are shown from N-terminus to C-terminus; all nucleic acid sequences disclosed herein are shown 5′->3′.

### EXAMPLES

The experiments shown herein clearly support SHP2 as a target whose inhibition provides a therapy option for kidney disease.

Experiments indicative for chronic kidney diseases were carried out in unilateral ureteral obstruction (UUO) mouse models (examples 1 and 2). Such treatment results in interstitial fibrosis and nephron damage associated with irreversible loss of function. Collagen expression is increased in UUO as a result of renal fibrosis, while alpha-smooth muscle actin (ASMA) expression is increased as a result of reduced renal function, or fibroblast expansion (see Chevalier et al, 2009).

Experiments indicative for acute kidney diseases were carried out in a uninephrectomy/renal ischemia reperfusion injury (uIRI) model (example 3), as e.g. disclosed in Skrypnyk et al (2013). As parameters, the albumin-to-creatinine ratio (uACR) and the expression of NHPS1 and NHPS2 was measured. uACR is a marker for albuminuria and hence indicates kidney injury, while the expression of the two podocyte markers Nephrin (NHPS1), which is an essential component of the glomerular slit diaphragm, and Podocin (NPHS2), which is a transmembrane protein involved in recruitment of nephrin at the slit diaphragm, and detects podocyte integrity.

The study was performed on male C57/B16J mice (age: 7-8 weeks) that were obtained from Charles River. Mice were anesthetized with continuous inhaled isoflurane, and the left ureter was exposed via a mid-abdominal incision. The mid-ureter was obstructed by two point ligation with silk sutures. The SHAM-operated mouse (n=6) underwent the same procedure except for the obstruction of the left ureter.

The mice were randomized into four groups (n=10 each group): UUO plus vehicle (10% EtOH/40% PEG/50% water), UUO plus 3 [mg/kg] SHP099, UUO plus 30 [mg/kg] SHP099. Mice were dosed bidaily with vehicle and SHP099. The forth group of mice with UUO was treated once daily with Tivozanib (technical control). At ten days after surgery, mice were euthanized, and kidneys were collected and divided in two parts.

One part was snap-frozen in liquid nitrogen for RNA analysis. The other part was stored in Davidson's fixative for the preparation of histological sections. Total RNA was isolated from parts of harvested kidneys. Kidney tissue was homogenized and RNA was obtained and transcribed to cDNA. Using TaqMan real time PCR renal mRNA expression of fibrotic markers was analyzed in kidney tissues. For the assessment of fibrosis on the protein level paraffin tissue sections were stained with alpha-smooth muscle actin (αSMA) and Sirius Red/Fast Green Collagen Stainings using standard procedures.

Quantitative measurements of alpha-smooth muscle actin (αSMA)-positive as well as Sirius Red (Collagen) positive areas within the kidneys were obtained by computer image analysis using the Axio Scan Z1 (Zeiss) microscope and the Zen software.

All data are expressed as means±S.D. Differences between groups were analyzed by one-way ANOVA with Dunnett's corrections for multiple comparisons. Statistical significance was defined as p<0.05.

The UUO enhanced the RNA expression of fibrotic markers (e.g. αSMA, Collagens) within the kidney. Treatment with SHP099 resulted in a dose-dependent reduction of αSMA and collagen RNA expression.

Histological stainings demonstrated an increase in αSMA and Collagen (SR/FG staining) content of obstructed kidneys in comparison to SHAM animals. Treatment with 3 [mg/kg] SHP099 led to a significant reduction of αSMA-positive area within the obstructed kidney. Collagen content was dose dependently and significantly reduced after treatment with both dosages of SHP099. Both parameters were significantly reduced by the treatment with Tivozanib (technical control).

### Example 1

In a Mouse 3 day unilateral ureter obstruction (UUO) model, the left ureter of a mouse was ligated, and immediately thereafter, treatment was initiated with the allosteric SHP2 inhibitor SHP099 (100 mg/kg; group 3), and the VEGF receptor tyrosine kinase inhibitor Tivozanib (3 mg/kg; group 4) as a non-SHP2 specific control, both twice daily. As a placebo, a solution without active ingredient was used (group 2), and a sham operated group with unligated kidney was used as a positive control (group 1)

| | animals | |
|---|---|---|
| group | (n) | treatment |
| 1 | 12 | sham operation with unligated kidney, |
| | | no further treatment |
| 2 | 12 | vehicle (10% Ethanol/40% Solutol/50% |
| | | Water), BID p.o. |
| 3 | 12 | 100 mg/kg SHP099 (10% Ethanol/40% |
| | | Solutol/50% Water), BID p.o. |
| 4 | 12 | 3 mg/kg Tivozanib (10% Ethanol/40% |
| | | Solutol/50% Water) QD p.o. |

The infiltration of macrophages in response to the renal injury was measured in a flow cytometry assay with detection antibodies binding to the pan-leukocyte marker CD45, as well as to the macrophage-specific antigen, F4/80.

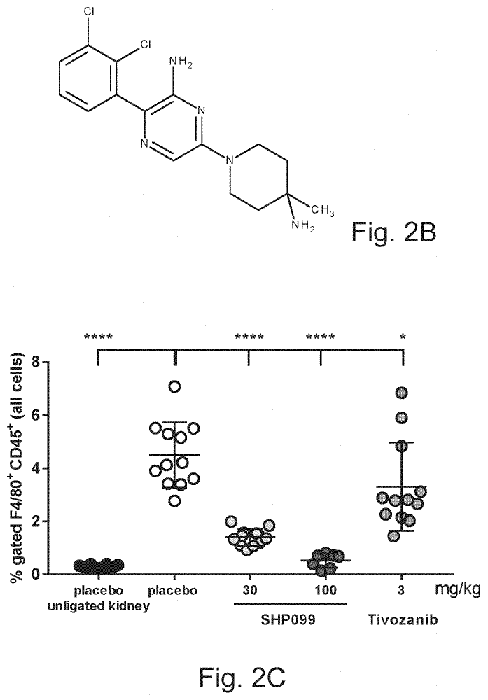

Results are shown in FIG. 2. A significant, dose-dependent reduction of macrophage infiltration in the SHP099 treated group could be determined, while the technical control Tivozanib (RTKi) showed weaker effects in comparison.

SHP099 has a MW 352 Da, a log D: 1.7, and SHP2 IC₅₀ of 0.071 μM. Results are shown in FIG. 2

### Example 2

In a Mouse 10 day unilateral ureter obstruction (UUO) model, the left ureter of a mouse was ligated, and immediately thereafter, treatment was initiated with the allosteric SHP2 inhibitor SHP099, and the tyrosine kinase inhibitor Tivozanib as a control, twice daily.

After 10 days, the weight of the kidneys was measured, and RNA-markers αSMA, Col1α1, Col3α1, Col4α1 were measured with RT PCR. Further, histological measurements of αSMA (Alpha smooth muscle actin) and Collagen (SR/FG) were carried out.

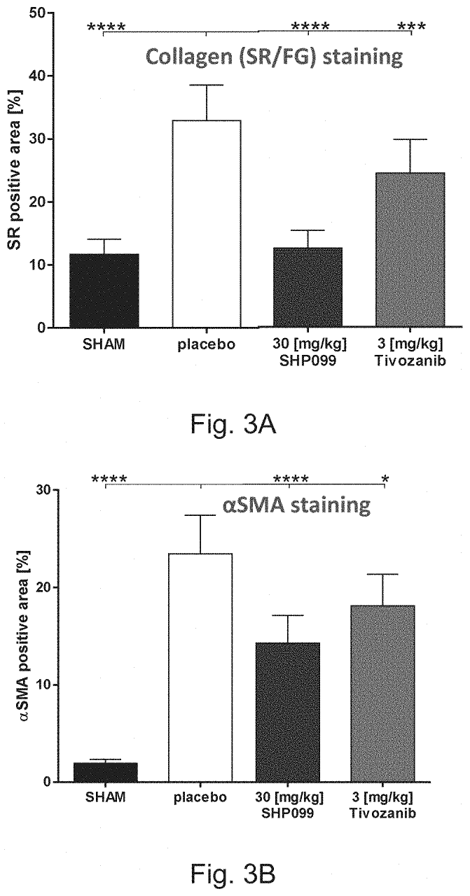

Histological results are shown in FIG. 3. A strong and significant reduction of the collagen deposition in kidneys from the SHP099 treated UUO mice could be observed, as compared to untreated or Tivozanib treated mice. Further, a strong and significant reduction of the aSMA-positive area in kidneys from SHP099 treated UUO mice could be observed.

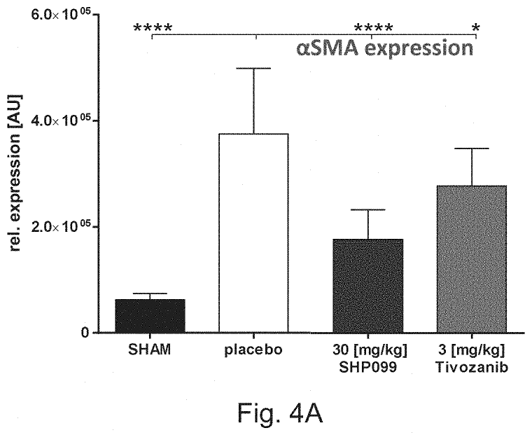

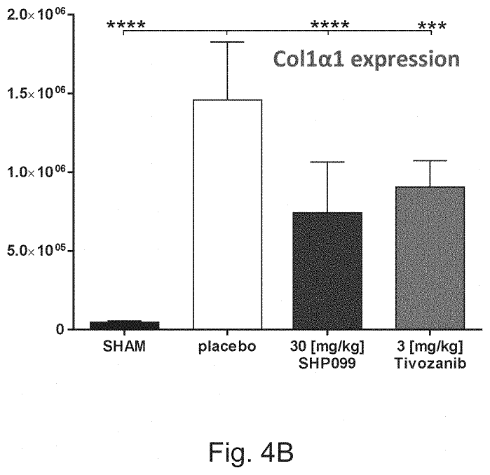

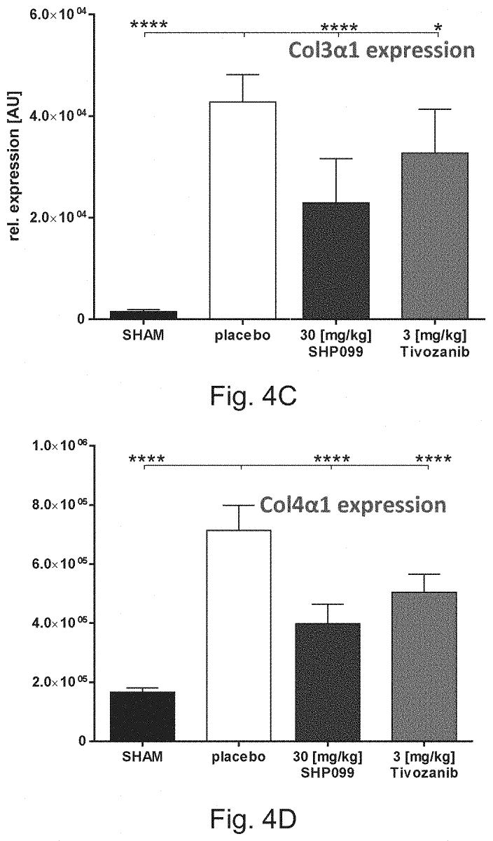

Results of the mRNA expression analysis are shown in FIG. 4. A strong and very significant reduction of the aSMA and Collagen 1a1, 3a1 and Collagen 4a1 mRNA expression in kidneys from SHP099 treated UUO mice was observed.

Results of the mRNA expression analysis are also shown in the following table as percentage values calculated vs. placebo.

| | αSMA | Col1α1 | Col3α1 | Col4α1 |
|---|---|---|---|---|
| SHAM | 17 | 3 | 3 | 23 |
| Placebo | 100 | 100 | 100 | 100 |
| 3 [mg/kg] | 81 | 77 | 82 | 85 |
| SHP099 | | | | |
| 30 [mg/kg] | 47 | 51 | 54 | 56 |
| SHP099 | | | | |
| Tivozanib | 64* | 62 | 76 | 71 |

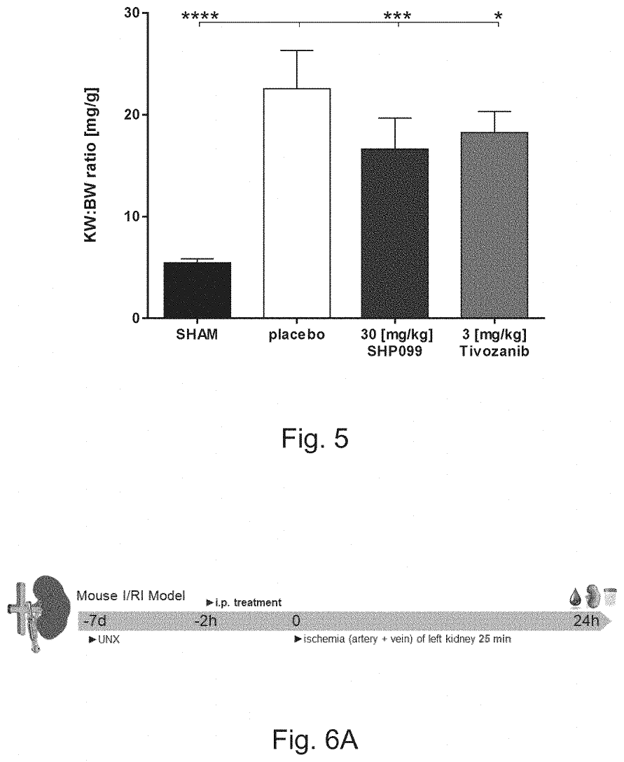

Results of the weight analysis are shown in FIG. 5. A strong and significant reduction of the ratio between kidney weight and body weight in the HP099 treated UUO mice was observed.

### Example 3

In a mouse renal ischemia reperfusion injury (IRI) model, uninephrectomy (UNX) with unilateral renal ischemia (RI), was carried out. Therein, one kidney is removed and the remaining kidney undergoes ischemia/reprefusion via renal artery claming, Animals were then treated according to the following groups:

| | animals | |
|---|---|---|
| group | (n) | treatment |
| 1 | 6 | sham operated animals (UNX only without |
| | | subsequent RI) with no further treatment |
| 2 | 12 | vehicle (10% Ethanol/40% Solutol/50% |
| | | water), p.o. BID |
| 3 | 12 | 30 [mg/kg] SHP099 p.o. BID |

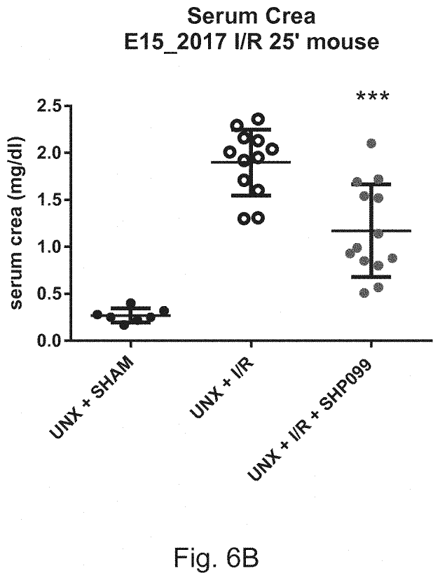

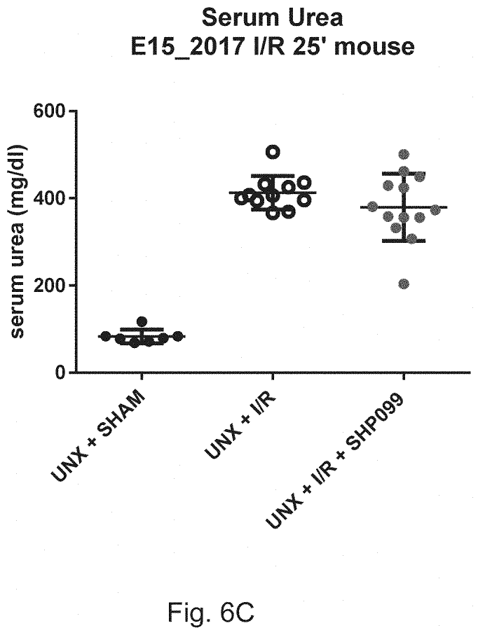

Results are shown in FIGS. 6B and C. In the animals treated with SHP099, a robust reduction of serum creatinine and a mild reduction of serum urea was detected.

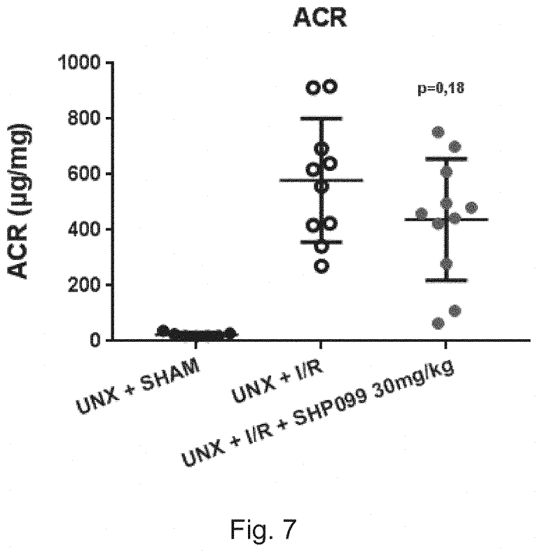

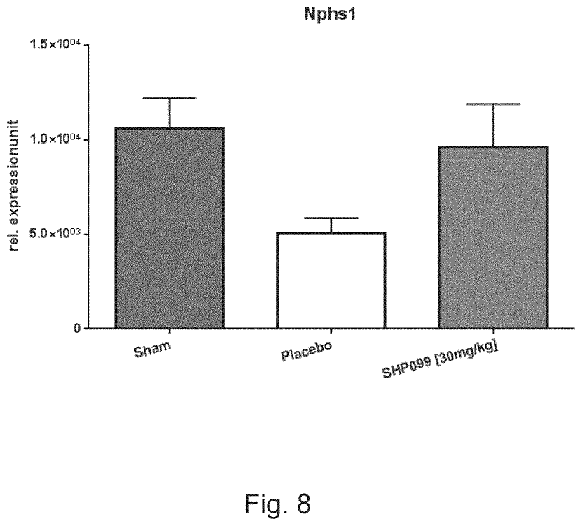

Further results are shown in FIGS. 7 and 8. A mild effect of treatment with SHP099 was found on the urine Albumin-to-Creatinine Ratio (uACR) was found in the uninephrectomy/ischemia reperfusion injury (uIRI) model. Further, a reproducible and strong effects on podocyte marker NHPS1 could be demonstrated (data not shown for NPHS2).

### FIGURES

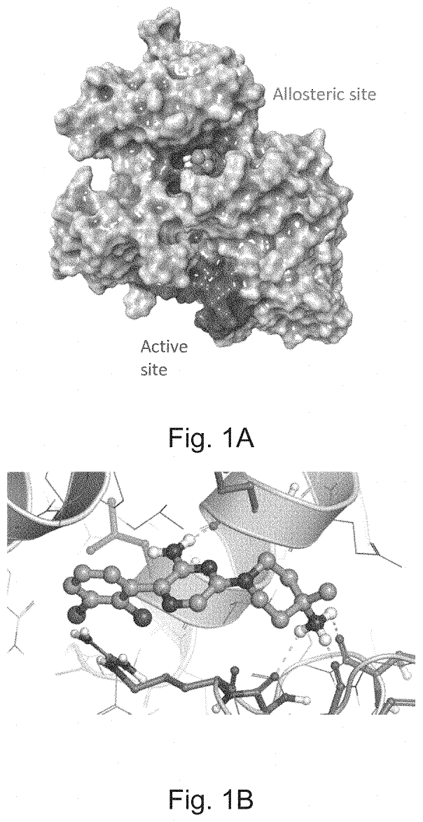

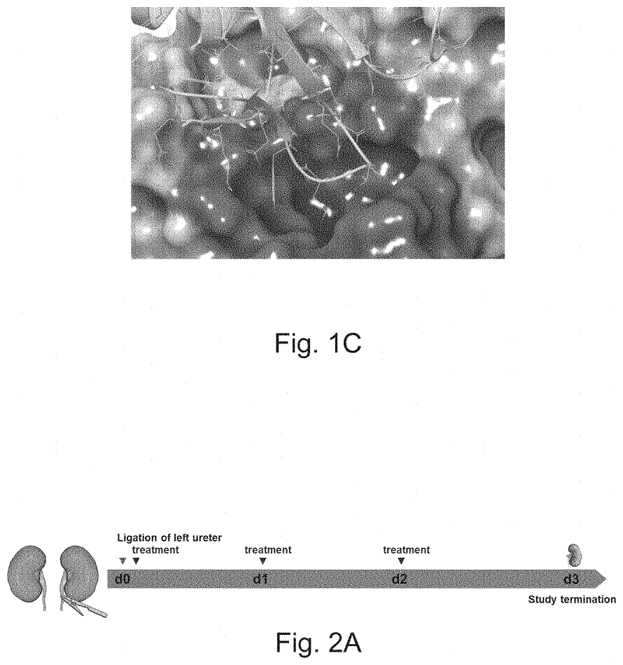

FIG. 1 shows a rendering of SHP2 with the allosteric binding site where SHP099 binds, and the active enzyme site. FIG. 1A shows the entire kinase, while FIG. 1B shows the allosteric binding site in more detail, and FIG. 1C shows the active site in more detail. The allosteric site is deep, allows lipophilic interaction and offers various options for other interactions. The active site is shallow, and does not allow lipophilic interactions because it is highly charged, hence making inhibition with organics small molecules difficult.

FIG. 2 shows the results of the experiments of example 1. FIG. 2A: Timeline of the experiments FIG. 2B: structure of SHP099. FIG. 2C: Results of the experiments

FIG. 3 shows the results of the histological experiments of example 2. FIG. 3A: results of the collagen (SR/FG) staining. FIG. 3B: results of the αSMA staining.

FIG. 4 shows the results of mRNA analysis experiments of example 2.

FIG. 5 shows the results of the weight analysis experiments of example 2.

FIG. 6 shows the results of the experiments of example 3. FIG. 6A: Timeline of the experiments FIG. 6B: Effect on Creatinin serum levels; FIG. 6C: Effect on Urea serum levels.

FIG. 7 shows results of the ACR screening in example 3

FIG. 8 shows the results of the NPHS1 expression analysis according to example 3.

### SEQUENCE LISTING

A sequence listing is enclosed which discloses the following sequences:

| No | Type | Sequence |
|---|---|---|
| 1 | SHP2 | MTSRRWFHPNITGVEAENLLLTRGVDGSFLARPSKSNPGDFTLSVRRNGAVTHIKIQNTG |
| | AA sequence | DYYDLYGGEKFATLAELVQYYMEHHGQLKEKNGDVIELKYPLNCADPTSERWFHGHLSGK |
| | Isoform 1 | EAEKLLTEKGKHGSFLVRESQSHPGDFVLSVRTGDDKGESNDGKSKVTHVMIRCQELKYD |
| | | VGGGERFDSLTDLVEHYKKNPMVETLGTVLQLKQPLNTTRINAAEIESRVRELSKLAETT |
| | | DKVKQGFWEEFETLQQQECKLLYSRKEGQRQENKNKNRYKNILPFDHTRVVLHDGDPNEP |
| | | VSDYINANIIMPEFETKCNNSKPKKSYIATQGCLQNTVNDFWRMVFQENSRVIVMTTKEV |
| | | ERGKSKCVKYWPDEYALKEYGVMRVRNVKESAAHDYTLRELKLSKVGQALLQGNTERTVW |
| | | QYHFRTWPDHGVPSDPGGVLDFLEEVHHKQESIMDAGPVVVHCSAGIGRTGTFIVIDILI |
| | | DIIREKGVDCDIDVPKTIQMVRSQRSGMVQTEAQYRFIYMAVQHYIETLQRRIEEEQKSK |
| | | RKGHEYTNIKYSLADQTSGDQSPLPPCTPTPPCAEMREDSARVYENVGLMQQQKSFR |
| 2 | SHP2 | MTSRRWFHPNITGVEAENLLLTRGVDGSFLARPSKSNPGDFTLSVRRNGAVTHIKIQNTG |
| | AA sequence | DYYDLYGGEKFATLAELVQYYMEHHGQLKEKNGDVIELKYPLNCADPTSERWFHGHLSGK |
| | Isoform 2 | EAEKLLTEKGKHGSFLVRESQSHPGDFVLSVRTGDDKGESNDGKSKVTHVMIRCQELKYD |
| | | VGGGERFDSLTDLVEHYKKNPMVETLGTVLQLKQPLNTTRINAAEIESRVRELSKLAETT |
| | | DKVKQGFWEEFETLQQQECKLLYSRKEGQRQENKNKNRYKNILPFDHTRVVLHDGDPNEP |
| | | VSDYINANIIMPEFETKCNNSKPKKSYIATQGCLQNTVNDFWRMVFQENSRVIVMTTKEV |
| | | ERGKSKCVKYWPDEYALKEYGVMRVRNVKESAAHDYTLRELKLSKVGQGNTERTVWQYHF |
| | | RTWPDHGVPSDPGGVLDFLEEVHHKQESIMDAGPVVVHCSAGIGRTGTFIVIDILIDIIR |
| | | EKGVDCDIDVPKTIQMVRSQRSGMVQTEAQYRFIYMAVQHYIETLQRRIEEEQKSKRKGH |
| | | EYTNIKYSLADQTSGDQSPLPPCTPTPPCAEMREDSARVYENVGLMQQQKSFR |
| 3 | SHP2 | MTSRRWFHPNITGVEAENLLLTRGVDGSFLARPSKSNPGDFTLSVRRNGAVTHIKIQNTG |
| | AA sequence | DYYDLYGGEKFATLAELVQYYMEHHGQLKEKNGDVIELKYPLNCADPTSERWFHGHLSGK |
| | Isoform 3 | EAEKLLTEKGKHGSFLVRESQSHPGDFVLSVRTGDDKGESNDGKSKVTHVMIRCQELKYD |
| | | VGGGERFDSLTDLVEHYKKNPMVETLGTVLQLKQPLNTTRINAAEIESRVRELSKLAETT |
| | | DKVKQGFWEEFETLQQQECKLLYSRKEGQRQENKNKNRYKNILPFDHTRVVLHDGDPNEP |
| | | VSDYINANIIMPEFETKCNNSKPKKSYIATQGCLQNTVNDFWRMVFQENSRVIVMTTKEV |
| | | ERGKSKCVKYWPDEYALKEYGVMRVRNVKESAAHDYTLRELKLSKVGQGNTERTVWQYHF |
| | | RTWPDHGVPSDPGGVLDFLEEVHHKQESIMDAGPVVVHCR |

### REFERENCES

The following articles are referred to in this specification. For enablement purposes of the present invention, the content thereof is incorporated herein by reference.
Lyo et al, Am J Physiol Gastrointest Liver Physiol, 2012 Oct. 15; 303(8):G894-903.
Liu et al., Atherosclerosis, 2006, 411-419.
Chihanga, Am J Physiol 2018, F154-F166.
Zhu et al, BMC nephrology, 2013, 14-21.
Saxton et al., EMBO J. 1997 May 1; 16(9):2352-64.
David et al., The Anatomical Record 293:2147-2153 (2010))
Wang et al, J Clin Invest. 2016; 126(6):2077-2092
Teng et al., Chin. Med. J. 128(9), 1196, May 5, 2015
Fodor et al., ACS Chem Biol. 2018 Mar. 16; 13(3):647-656
Chio et al, Biochemistry 2015, 54, 497-504
Chen, & Erlanger, Immunol Lett 5; 88(2): 87 (2003)
Berguig et al., Mol Ther May; 23(5):907-17 (2015)
Köhler & Milstein, Nature 256, 495-497 (1975)
Jinek et al., Science 17; 337(6096): 816-21 (2012),
Yin et al., Nature Biotechnology 34, 328-333. (2016)
Zetsche et al., Cell 22; 163(3):759-71 (2015)
Blind & Blank, Molecular Therapy Nucleic Acids 4, e223 (2015)
Thiel & Giangrande, Ther Deliv. 1(6):849-61 (2010)
Xu & Wang, Asian Journal of Pharmaceutical Sciences 10 (1), 1-12 (2015)
Xie et al., J Med Chem 2017, 60, 10205-10219
LaRochelle et al, Bioorganic & Medicinal Chemistry 25 (2017) 6479-6485
Chevalier et al., Kidney Int. 2009 June; 75(11):1145-52.
Skrypnyk et al. J Vis Exp. 2013 Aug. 9; (78)

1. A method for treating or preventing a kidney disease, comprising administering to a subject in need thereof an effective amount of an inhibitor or antagonist of SHP2.

2. The method of claim 1, wherein the inhibitor or antagonist of SHP2 is an allosteric inhibitor or antagonist.

3. The method of claim 1, wherein the inhibitor or antagonist of SHP2 is a monoclonal antibody, or a target-binding fragment or derivative thereof retaining target binding capacities, or an antibody mimetic, which specifically binds to the SHP2 protein.

4. The method of claim 1, wherein the inhibitor of SHP2 comprises a first nucleic acid molecule that specifically binds to a second nucleic acid molecule, which second nucleic acid molecule encodes for the SHP2 protein.

5. The method of claim 1, wherein the inhibitor or antagonist of SHP2 is an aptamer that specifically binds to the SHP2 protein.

6. The method of claim 1, wherein the inhibitor or antagonist of SHP2 is a small molecule that specifically binds to one or more isoforms of the SHP2 protein.

7. The method of claim 1, wherein the inhibitor of SHP2 can be found by means of a SHP2 inhibition assay.

8. The method of claim 3, wherein the SHP2 protein to which the antibody, fragment or derivative, or antibody mimetic binds comprises a sequence comprised in SEQ ID No 1.

9. The method of claim 6, wherein the nucleic acid encoding the SHP2 protein comprises a sequence comprised in SEQ ID No 2.

10. (canceled)

11. A method for treating or preventing a kidney disease, comprising administering to a subject in need thereof an effective amount of a pharmaceutical composition comprising an inhibitor or antagonist of SHP2.

12. (canceled)

13. A method for treating or preventing a kidney disease, comprising administering to a subject in need thereof an effective amount of a pharmaceutical combination comprising an inhibitor or antagonist of SHP2 and one or more therapeutically active compounds.

14. A method for identifying a compound for use in the treatment and/or prevention of a patient suffering from, at risk of developing, and/or being diagnosed for a kidney disease, which method comprises the screening of one or more test compounds in a SHP2 inhibition assay.

15. The method of claim 14, further comprising a prior step of creation and/or provision of a library of test compounds.

16. A method for determining whether a human or animal subject is suitable of being treated with an antagonist or inhibitor of SHP2, said method comprising
providing a tissue or liquid sample from said subject, and
determining whether or not said sample is characterized by expression or overexpression of SHP2.

17. The method according to claim 16, wherein the expression of SHP2 is determined
on an mRNA level;
on a protein level; and/or
on a genomic level.

18. The method of claim 16, further comprising using a companion diagnostic, which companion diagnostic comprises at least one agent selected from the group consisting of
a nucleic acid probe or primer capable of hybridizing to a nucleic acid (DNA or RNA) that encodes an SHP2 protein,
an antibody that is capable of binding to a SHP2 protein, and
an aptamer that is capable of binding to a SHP2 protein.

19. The method of claim 5, wherein the SHP2 protein to which the aptamer or binds comprises a sequence comprised in SEQ ID No 1.

20. The method of claim 6, wherein the SHP2 protein to which the small molecule binds comprises a sequence comprised in SEQ ID No 1.
